(19) United States
(12) Patent Application Publication
(10) Pub. No.: US 2024/0358637 A1
(43) Pub. Date: Oct. 31, 2024

(54) SPRAY APPLICATION OF WATER SOLUBLE ACTIVES TO DRINKING VESSELS AND OTHER OBJECTS

(71) Applicants: Gordon Brittain Whelpley, III, Springfield, MA (US); Jonathan Lafrenaye, Springfield, MA (US)

(72) Inventors: Gordon Brittain Whelpley, III, Springfield, MA (US); Jonathan Lafrenaye, Springfield, MA (US)

(21) Appl. No.: 18/648,010

(22) Filed: Apr. 26, 2024

Related U.S. Application Data
(60) Provisional application No. 63/568,872, filed on Mar. 22, 2024.
(60) Provisional application No. 63/462,886, filed on Apr. 28, 2023.

(51) Int. Cl. A61K 9/00; A61K 31/00; A61K 47/26; A61K 47/38

(52) U.S. Cl. CPC A61K 9/0095; A61K 31/658; A61K 47/26; A61K 47/38

(57) ABSTRACT

Disclosed is a process of preparing a coated surface, the process comprising applying a composition to the surface, wherein the composition comprises about 0.001% to about 2% w/w of a cannabinoid, at least one aqueous stabilizing agent; at least one cellulose derivative; and at least one taste-masking agent.

### RELATED APPLICATIONS

This application claims the benefit of priority to U.S. Provisional Patent Application Nos. 63/568,872, filed Mar. 22, 2024; and 63/462,886, filed Apr. 28, 2023, the contents of each of which are incorporated herein by reference in their entirety.

### BACKGROUND

Cannabinoids are a class of chemicals that can act on cannabinoid receptors. Cannabinoid receptor ligands include endocannabinoids, which can be found naturally occurring in humans and other animals, phytocannabinoids, which can be found in cannabis and other plants, other plants, and lichens, and synthetic cannabinoids. Cannabinoids include tetrahydrocannabinol (THC), such as delta-9-tetrahydrocannabinol, and cannabidiol (CBD). At least 85 different cannabinoids have been isolated from cannabis.

The consumption of water-soluble actives, such as cannabinoids, are becoming increasingly popular. However, traditional methods of consumption, such as pills or liquids, can be inconvenient to dose on the go and lend themselves to potential user error in the dosing procedure. Therefore, there is a need for a more convenient, accessible, and safe method of consuming these actives.

### SUMMARY

One aspect of the present invention provides a process of preparing a coated surface, the process comprising applying a composition to the surface, wherein the composition comprises:
(i) about 0.001% to about 2% w/w of a cannabinoid.
(ii) at least one aqueous stabilizing agent;
(iii) at least one cellulose derivative; and
(iv) at least one taste-masking agent.

Another aspect of the present invention provides a process of preparing a cannabinoid containing beverage, the process comprising adding a liquid to a surface coated drinking vessel.

### BRIEF DESCRIPTION OF THE DRAWINGS

FIG. 1 is a view of precision spray application of the composition to a drinking vessel.

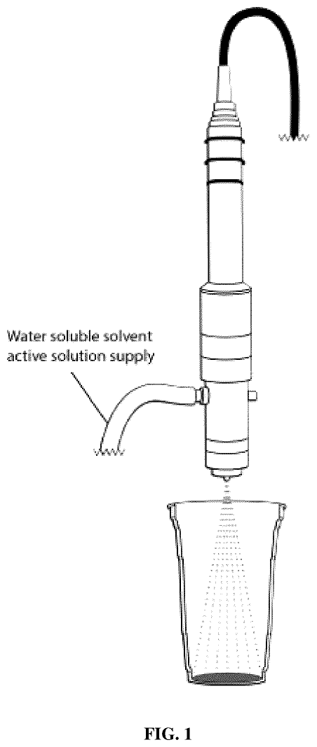

FIG. 2 is a view of the coating on the bottom of the drinking vessel following spray application.

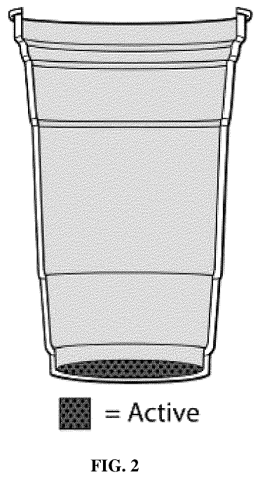

### DETAILED DESCRIPTION

The present invention relates to a process for applying a water-soluble active, such as a cannabinoid, to a drinking vessel, such as a cup or can, or other object, such as a straw. The process includes preparing a solution of the active, and applying the solution to the object using a precision spray application system. The solvent evaporates, leaving the active on the surface of the object, which can then be dissolved in a liquid for consumption.

Provided herein is a convenient and palatable way to consume water-soluble actives, such as a cannabinoid. The spray application allows for precise control of the amount of active applied, and the solvent evaporates, leaving only the active on the surface of the object, ensuring that it is available for dissolution in a liquid for consumption. The active also possesses good solubility properties. The suspension disclosed herein provides the advantage of high solid content without becoming too viscous, thereby provided a superior medium for spray application.

### Exemplary Embodiments of the Invention

One aspect of the present invention provides a process of preparing a coated surface, the process comprising applying a composition to the surface, wherein the composition comprises:
(i) about 0.001% to about 2% w/w of a cannabinoid.
(ii) at least one aqueous stabilizing agent;
(iii) at least one cellulose derivative; and
(iv) at least one taste-masking agent.

One aspect of the present invention provides a process of preparing a coated surface, the process comprising applying a composition to the surface, wherein the composition comprises:
(i) about 0.001% to about 10% w/w of a cannabinoid;
(ii) at least one aqueous stabilizing agent; and
(iv) at least one taste-masking agent.

One aspect of the present invention provides a process of preparing a coated surface, the process comprising applying a composition to the surface, wherein the composition comprises:
(i) about 0.001% to about 2% w/w of a cannabinoid;
(iii) at least one cellulose derivative; and
(iv) at least one taste-masking agent.

In some embodiments, the composition comprises about 0.001% to about 1% w/w of the cannabinoid.

In some embodiments, the composition comprises about 0.1% to about 0.5% w/w of the cannabinoid.

In some embodiments, the composition comprises about 0.5% to about 1% w/w of the cannabinoid.

In some embodiments, the composition comprises about 1.0% to about 1.5% w/w of the cannabinoid.

In some embodiments, the composition comprises about 1.5% to about 2% w/w of the cannabinoid.

In some embodiments, the composition comprises about 0.1% to about 1% w/w of the cannabinoid.

In some embodiments, the composition comprises about 0.1% to about 0.2% w/w, about 0.2% to about 0.3% w/w, about 0.3% to about 0.4% w/w, about 0.4% to about 0.5% w/w, about 0.5% to about 0.6% w/w, about 0.6% to about 0.7% w/w, about 0.7% to about 0.8% w/w, about 0.8% to about 0.9% w/w, or about 0.9% to about 1.0% w/w of the cannabinoid.

In some embodiments, the composition comprises about 1% to about 2% w/w of the cannabinoid.

In some embodiments, the composition comprises about 1% to about 1.1% w/w, about 1.1% to about 1.2% w/w, about 1.2% to about 1.3% w/w, about 1.3% to about 1.4% w/w, about 1.4% to about 1.5% w/w, about 1.5% to about 1.6% w/w, about 1.6% to about 1.7% w/w, about 1.7% to about 1.8% w/w, about 1.8% to about 1.9% w/w, or about 1.9% to about 2.0% w/w of the cannabinoid.

In some embodiments, the cannabinoid is a cannabinol or cannabidiol.

In some embodiments, the cannabinoid is delta-9-tetrahydrocannabinol.

In some embodiments, the at least one aqueous stabilizing agent is selected from agar, chitosan, hyaluronic acid, carrageenan, gellan gum, xanthan gum, hydroxypropylmethylcellulose, hydroxypropylcellulose hydroxyethylcellulose, carboxymethylcellulose, methylcellulose and microcrystalline cellulose.

In some embodiments, the at least one aqueous stabilizing agent is selected from agar, chitosan, hyaluronic acid, carrageenan, gellan gum, xanthan gum, hydroxypropylmethylcellulose, carboxymethylcellulose, methyl cellulose and hydroxypropylcellulose.

In some embodiments, the at least one aqueous stabilizing agent is selected from agar, chitosan, hyaluronic acid, carrageenan, gellan gum, and xanthan gum.

In some embodiments, the at least one aqueous stabilizing agent is selected from xanthan gum and hydroxypropylcellulose.

In some embodiments, the at least one aqueous stabilizing agent is xantham gum.

In some embodiments, the at least one aqueous stabilizing agent is hydroxypropylcellulose.

In some embodiments, the composition comprises about 5% to about 10% w/w of the at least one aqueous stabilizing agent.

In some embodiments, the composition comprises about 5% to about 6% w/w, about 6% to about 7% w/w, about 7% to about 8% w/w, about 8% to about 9% w/w, or about 9% to about 10% w/w of the at least one aqueous stabilizing agent.

In some embodiments, the at least at least one cellulose derivative is selected from hydroxypropylmethylcellulose, hydroxypropylcellulose hydroxyethylcellulose, carboxymethylcellulose, methylcellulose, and microcrystalline cellulose.

In some embodiments, the at least at least one cellulose derivative is selected from hydroxypropylmethylcellulose and hydroxypropylcellulose.

In some embodiments, the at least at least one cellulose derivative is hydroxypropylmethylcellulose.

In some embodiments, the composition comprises about 0.5% to about 1% w/w of the at least at least one cellulose derivative.

In some embodiments, the composition comprises about 0.5% to about 0.6% w/w, about 0.6% to about 0.7% w/w, about 0.7% to about 0.8% w/w, about 0.8% to about 0.9% w/w, or about 0.9% to about 1.0% w/w of the at least at least one cellulose derivative.

In some embodiments, the at least one taste-masking agent is selected from a sweetener and a flavoring agent.

In some embodiments, the sweetener is a carbohydrate.

In some embodiments, the sweetener is selected from a monosaccharide, a disaccharide, and a polysaccharide.

In some embodiments, the disaccharide is sucrose.

In some embodiments, the monosaccharide is glucose.

In some embodiments, the sweetener is a polyol.

the polyol is selected from mannitol, xylitol, and sorbitol.

In some embodiments, the flavoring agent is citric acid.

In some embodiments, the at least one taste-masking agent comprises about 10% to about 20% w/w of the composition.

In some embodiments, the composition comprises about 10% to about 11% w/w, about 11% to about 12% w/w, about 12% to about 13% w/w, about 13% to about 14% w/w, about 14% to about 15% w/w, about 15% to about 16% w/w, about 16% to about 17% w/w, about 17% to about 18% w/w, about 18% to about 19% w/w, or about 19% to about 20% w/w of the at least one taste-masking agent.

In some embodiments, the composition further comprises (v) at least one disintegrant, e.g. carboxymethylcellulose or modified food starch; (vi) at least one filler, e.g. mannitol or microcrystalinecellulose; and/or (vii) at least one pH adjuster, e.g. citric acid, sodium citrate, monopotassium phosphate, dipotassium phosphate, tripotassium phosphate, sodium hexametaphosphate, malic acid, lactic acid, ascorbic acid, or sodium ascorbate.

In some embodiments, an aqueous suspension comprising the composition is applied to the surface.

In some embodiments, the aqueous suspension further comprises ethanol, glycerol, and/or propylene glycol.

In some embodiments, the aqueous suspension is dried following application, thereby forming a solid coating on the surface.

In some embodiments, the aqueous suspension is applied by spray drying.

In some embodiments, the aqueous suspension is applied by a spray application system, such as an aerosol can or a pump sprayer.

In some embodiments, the aqueous suspension is applied by a precision spray application system, such as an aerosol can or a pump sprayer.

In some embodiments, the surface is a surface of drinking vessel, i.e. a glass cup, metal cup, paper cup, or plastic cup.

In some embodiments, the surface is the interior surface of a drinking vessel.

In some embodiments, the surface is a surface of an accessory to a drinking vessel.

In some embodiments, the surface is the interior surface of an accessory to a drinking vessel.

In some embodiments, the accessory to a drinking vessel is a straw.

Another aspect of the present invention provides a process of preparing a cannabinoid containing beverage, the process comprising adding a liquid to a surface coated drinking vessel described herein.

In some embodiments, the interior surface of the drinking vessel is surface coated.

In some embodiments, the liquid is water.

A further aspect of the present invention provides a process of preparing the aqueous suspension of the present invention, the process comprising:
(i) added the at least one aqueous stabilizing agent, the at least one cellulose derivative and the at least one taste-masking agent to water;
(ii) mixing the product of step (i) and heating the mixture to a suitable temperature; and
(iii) adding the cannabinoid.

In some embodiments, the at least one aqueous stabilizing agent is hydroxypropylcellulose; the at least one cellulose derivative is hydroxypropylmethylcellulose; and the at least one taste masking agent is sucrose.

In some embodiments, further comprising (iv) adding a second aqueous stabilizing agent.

In some embodiments, the second aqueous stabilizing agent is xantham gum.

In some embodiments, the second aqueous stabilizing agent is methylcellulose.

In some embodiments, the suitable temperature is 45-55° C.

In some embodiments, the aqueous suspension produced is a homogenized solution.

A further aspect of the present invention provides a process for applying a cannabinoid to a drinking vessel, comprising applying an aqueous suspension of the present inventions by spray drying, to the drinking vessel.

A further aspect of the present invention provides a process for applying a cannabinoid to a drinking vessel, comprising (i) applying an aqueous suspension or a solution of the present invention by spray drying, to the drinking vessel; and (ii) allowing the solvent to evaporate, leaving the cannabinoid on the surface of the drinking vessel.

### Definitions

The term “cannabinoid” as used herein includes phytocannabinoids, endogenous cannabinoids, and synthetic cannabinoids. For example, a cannabichromene (CBC), annabichromevarin (CBCV), cannabidiol (CBD), cannabidiolic acid (CBDA), cannabidivarin (CBDV), cannabidivarinic acid (CBDVA), cannabigerol (CBG), Cannabigerivarin (CBGV), cannabicyclol (CBL), cannabinol (CBN), cannabinovarin (CBNV), cannabinorcin (CBO), delta-9-tetrahydrocannabinol (THC), tetrahydrocannabinolic acid (THCA), tetrahydrocannabivarin (THCV), and tetrahydrocannabivarinic acid (THCVA).

The term “spray drying” as used herein refers to any process for forming a dry powder from a liquid, slurry, emulsion, suspension, or paste by drying with a gas, e.g. air or nitrogen.

### EXAMPLES

The invention is further described in the following examples, which do not limit the scope of the invention described in the claims.

### Example 1. Composition Used in Spray Drying Process

The composition applied to the surface comprises (i) cannabinoid; (ii) at least one aqueous stabilizing agent; (iii) at least one cellulose derivative; and (iv) at least one taste-masking agent.

Optional components include (v) at least one disintegrant, e.g. carboxymethylcellulose or modified food starch; (vi) at least one filler, e.g. mannitol or microcrystalinecellulose; and/or (vii) at least one pH adjuster, e.g. citric acid or sodium citrate.

### Example 2. Exemplary Spray Application Process

1) 7.5% w/v HPC (Klucel nutra W grade HPC, a highly substituted hydroxypropylcellulose derivative. This particular lot has a pH of 6.2, a hydroxypropyl content of 3.8M/74.9% substitutions, and a 10% Viscosity, water at STP of 383 cP) with 10% w/v sugar (sucrose) in a solution of 50% EtOH was heated to 55° C. or until a cloud-point is noticed.
2) 0.5% w/v Methocel F50, a low viscosity hydroxypropylmethylcellulose was slowly added.
3) With agitation, cold water was slowly added to reach a final volume, providing a solution of <40% EtOH v/v, with 5% HPC/0.5% HPMC/10% sucrose w/w. The HPC solution is ⅔ of the final volume, the HPMC is added with the final volume of cold water (the w/w of the final dry above is 7.14% HPC/0.71% HPMC/14.29% sucrose).

An alternative approach is to grind the powders together, add to the final solution at 45-55° C., and allow to slow cool with minor agitation.

4) The cannabinoid, filler(s) and/or disintegrant(s) were slowly added to the solution with agitation after forming the stabilized excipient described above.

### Cannabinoid: Up to 2% w/w.

Disintegrants (about 1-2%, 2-3%, 3-4% or 2-4% w/w): Carboxymethylcellulose, 2-4% w/w (also has been used as thickener); pre-gelatinized Modified food starch (low molecular weight) ClearJel, 1-2% w/w.
Filler ingredients (about 10-20% or 15-40% w/w): Mannitol, 15-40% w/w; Microcrystalinecellulose, 10-20% w/w.
5) After the solutions is mixed thoroughly, a small amount (<0.15%) of A4C Methylcellulose is optionally added to further stabilize the solution. Citric Acid and Sodium Citrate are also optionally added to balance pH. Without these pH adjusters the pH of the solution falls between 6.25-6.88.
6) The solution is then passed through an atomized pressure sprayer to spray the solution on to a surface, e.g. the surface of a drinking vessel, which provides a dry coating on the surface.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A process of preparing a coated surface, the process comprising applying a composition to the surface, wherein the composition comprises:
(i) about 0.001% to about 2% w/w of a cannabinoid.
(ii) at least one aqueous stabilizing agent;
(iii) at least one cellulose derivative; and
(iv) at least one taste-masking agent.

2. The process of claim 1, wherein the composition comprises about 0.001% to about 1% w/w of the cannabinoid.

3. The process of claim 1, wherein the composition comprises about 0.1% to about 0.5% w/w, about 0.5% to about 1% w/w, about 1% to about 1.5% w/w, or about 1.5% to about 2% w/w of the cannabinoid.

4-7. (canceled)

8. The process of claim 1, wherein the composition comprises about 0.1% to about 0.2% w/w, about 0.2% to about 0.3% w/w, about 0.3% to about 0.4% w/w, about 0.4% to about 0.5% w/w, about 0.5% to about 0.6% w/w, about 0.6% to about 0.7% w/w, about 0.7% to about 0.8% w/w, about 0.8% to about 0.9% w/w, about 0.9% to about 1.0% w/w, about 1% to about 1.1% w/w, about 1.1% to about 1.2% w/w, about 1.2% to about 1.3% w/w, about 1.3% to about 1.4% w/w, about 1.4% to about 1.5% w/w, about 1.5% to about 1.6% w/w, about 1.6% to about 1.7% w/w, about 1.7% to about 1.8% w/w, about 1.8% to about 1.9% w/w, or about 1.9% to about 2.0% w/w of the cannabinoid.

9-10. (canceled)

11. The process of claim 1, wherein the cannabinoid is delta-9-tetrahydrocannabinol.

12. The process of claim 1, wherein the at least one aqueous stabilizing agent is selected from agar, chitosan, hyaluronic acid, carrageenan, gellan gum, xanthan gum, methylcellulose, hydroxypropylmethylcellulose, hydroxypropylcellulose, and carboxymethylcellulose.

13-14. (canceled)

15. The process of claim 1, wherein the at least at least one cellulose derivative is selected from hydroxypropylmethylcellulose, hydroxypropylcellulose hydroxyethylcellulose, carboxymethylcellulose, methylcellulose, and microcrystalline cellulose.

16-17. (canceled)

18. The process of claim 1, wherein the at least one taste-masking agent is selected from a sweetener and a flavoring agent.

19. (canceled)

20. The process of claim 18, wherein the sweetener is selected from a monosaccharide, a disaccharide, and a polysaccharide.

21-24. (canceled)

25. The process of claim 18, wherein the at least one taste-masking agent comprises about 10% to about 20% w/w of the composition.

26. The process of claim 1, wherein an aqueous suspension comprising the composition is applied to the surface.

27. The process of claim 26, wherein the aqueous suspension further comprises ethanol, glycerol, and/or propylene glycol.

28. The process of claim 26, wherein the aqueous suspension is dried following application, thereby forming a solid coating on the surface.

29. The process of claim 26, wherein the aqueous suspension is applied by spray drying.

30. The process of claim 26, wherein the surface is an interior surface of a drinking vessel or an interior surface of an accessory to a drinking vessel.

31. (canceled)

32. A process of preparing a cannabinoid containing beverage, the process comprising adding a liquid to the drinking vessel or the accessory to a drinking vessel of claim 30.

33. The process of claim 32, wherein the liquid is water.

34-36. (canceled)

37. A process of preparing the aqueous suspension of claim 26, the process comprising:
(i) adding the at least one aqueous stabilizing agent, the at least one cellulose derivative and the at least one taste-masking agent to water;
(ii) mixing the product of step (i) and heating the mixture to a suitable temperature; and
(iii) adding the cannabinoid,
wherein the aqueous solution produced is a homogenized solution.

38. The process of claim 37, wherein the at least one aqueous stabilizing agent is hydroxypropylcellulose; the at least one cellulose derivative is hydroxypropylmethylcellulose; and the at least one taste masking agent is sucrose.

39. The process of claim 37, further comprising (iv) adding a second aqueous stabilizing agent.

40-42. (canceled)
